(19) United States
(12) Patent Application Publication
(10) Pub. No.: US 2024/0423635 A1
(43) Pub. Date: Dec. 26, 2024

(54) LEFT ATRIAL APPENDAGE OCCLUSION METHODS AND DEVICES

(71) Applicant: Maurice Buchbinder, San Diego, CA (US)

(72) Inventor: Maurice Buchbinder, San Diego, CA (US)

(21) Appl. No.: 18/737,476

(22) Filed: Jun. 7, 2024

Related U.S. Application Data
(63) Continuation-in-part of application No. 16/629,332, filed on Jan. 8, 2020, now Pat. No. 12,016,569.
(63) Continuation of application No. 18/124,534, filed on Mar. 21, 2023.
(60) Provisional application No. 62/530,669, filed on Jul. 10, 2017.
(60) Provisional application No. 62/682,443, filed on Jun. 8, 2018.
(60) Provisional application No. 63/322,048, filed on Mar. 21, 2022.

(51) Int. Cl. A61B 17/12

(52) U.S. Cl. CPC A61B 17/12177; A61B 17/12109; A61B 2017/12081

(57) ABSTRACT

Left atrial occlusion devices and methods of occluding a left atrial appendage. The left atrial occlusion devices can be carried by a flexible elongate member, have an inflatable member, and have a plurality of flexible elongate implant members forming a distally open cage configuration when expanded. The implant members have a flexible format to allow them to conform to the anatomy of the left atrial appendage. The distal ends of the flexible elongate member can optionally be atraumatic.

### CROSS REFERENCE TO RELATED APPLICATIONS

This application incorporates by reference herein PCT publication WO 2019/014219, U.S. Patent Application No. 63/322,048, filed Mar. 21, 2022, and U.S. patent application Ser. No. 16/629,332 filed Jan. 8, 2020, as though set forth herein.

### BACKGROUND OF THE INVENTION

### Field of the Invention

The invention related to left atrial appendage closure devices and methods of employing them.

### Background Art

The left atrial appendage (“LAA”) is a small sac in the muscle wall of the left atrium. It is unclear what function, if any, the LAA performs. In normal hearts, the heart contracts with each heartbeat, and the blood in the left atrium and LAA is squeezed out of the left atrium into the left ventricle.

Atrial fibrillation (AF) is the irregular, chaotic beating of the upper chambers of the heart. Electrical impulses that control the heartbeat do not travel in an orderly fashion through the heart. Instead, many impulses begin at the same time and spread through the atria. The fast and chaotic impulses do not give the atria time to contract and/or effectively squeeze blood into the ventricles. As a result, the blood is not squeezed from the LAA in regular heartbeats. Because the LAA is a little pouch, blood collects there and can form clots in the LAA and atria. When blood clots are pumped out of the LAA, and then out of the heart, they can cause a stroke.

It is estimated that AF patients have five times the stroke risk of patients without AF. Most AF patients, regardless of the severity of their symptoms or frequency of episodes, require treatment to reduce the risk of stroke. In non-valvular AF, over 90% of stroke-causing clots that come from the left atrium are formed in the LAA.

The most common treatment for stroke risk reduction in patients with AF is blood-thinning therapy with oral anti-coagulants. Oral anti-coagulants effectively reduce the risk of cardioembolic stroke and are the most commonly used treatments in at-risk patients with non-valvular atrial fibrillation. Many patients have concerns about, or dislike, taking oral anti-coagulants. Some of the reasons for this are: Frequent blood draws are needed to measure the patient's international normal ratio (INR), or clotting time. The tests are needed to make sure the patient takes the right amount of medication; while taking warfarin, you need to limit your intake of certain foods that contain vitamin K; the risk of bleeding is higher while taking oral anti-coagulants; and some patients do not tolerate medical therapy.

While it is common to perform a LAA closure in AF patients, a LAA closure can also benefit patients who need heart surgery, or other risk factors for a stroke.

There has thus been a desire to attempt to filter, occlude and/or isolate the LAA to prevent clots from forming therein, which can be subsequently released from the LAA and cause a stroke. It is also desirable to occlude the LAA to isolate blood clots that may already be forming in the left atrial appendage.

There are devices on the market that are adapted to filter and/or occlude the LAA to reduce the likelihood of stroke. For example, the Watchman™ device (FDA approved in 2015) is implanted in the left atrial appendage, and initially acts as a filter between the LAA and the atria to prevent clots from being released from the LAA. Over time, cells grow over the device, effectively sealing off the LAA from the atrium. US Publication 2016/0058539, including all of its methods of delivering an occlusion device to the LAA, are incorporated by reference herein.

The anatomy of the LAA is not consistent from one patient to the next. The LAA can have substantially different sizes from one patient to the next. The opening can also be highly irregular. As a result, current approaches require many differently sized and shaped implants available for implantation depending on the anatomy of a particular patient, which is generally assessed prior to implantation using imaging techniques, such as ultrasound imaging techniques (e.g., TEE) and computerized tomography (CT). There is a need for modified and improved occlusion devices and steps to more efficiently determine an appropriate size of the implant based on the patient's anatomy.

### BRIEF SUMMARY OF THE INVENTION

The present invention solves these needs by providing a system for deploying a left atrial appendage occlusion device that includes a sheath, a delivery catheter that has a lumen, a balloon attached to a distal end of the delivery catheter and in fluid communication with the lumen; the balloon having a preformed shape when inflated. The balloon is constructed of a compliant material. The system also includes a left atrial appendage occlusion device with an endoskeleton constructed of a flexible material and having barbs, as well as a barrier layer comprising a knit fabric. The endoskeleton is configured to undergo plastic deformation from a first, compact form into a second, expanded form when the balloon expands; remaining in the second expanded form when the balloon deflates.

In another embodiment the flexible material is a stainless steel. It can have an elastic modulus between 100-310 GPA. The endoskeleton can be constructed from a hypotube. The hypotube can have a pattern cut into it to allow the hypotube to expand outward under pressure from the balloon.

In one embodiment the hypotube has a proximal region and a distal region. More material can be removed from the proximal region than from the distal region. In some embodiments the flexible material is semi-compliant.

In some embodiments the system includes a balloon catheter. The distal balloon end is attached to the balloon catheter, and a proximal balloon end is attached to the distal end of the delivery catheter, and the balloon catheter is configured to move laterally with respect to delivery catheter.

In some embodiments the left atrial appendage occlusion device comprises a central valve. In others, the balloon is constructed of a polyurethane. In one embodiment the barbs are only located in the distal half of the endoskeleton. In another, the barbs are configured to open outward when the endoskeleton is in the expanded form.

In one embodiment the endoskeleton further comprises a ring, the ring having a lumen with an inner diameter, the inner diameter of the ring being slightly larger than the outer diameter of the balloon before inflation. In another embodiment the inner diameter of the ring is smaller than the outer diameter of the catheter.

In one embodiment the invention is a method of occluding a left atrial appendage (“LAA). The method includes the steps of delivering a system for deploying a left atrial appendage occlusion device to the left atrial appendage, the system including a delivery catheter with a balloon attached to a distal end of the delivery catheter; the balloon having a preformed shape when inflated; wherein the balloon is constructed of a compliant material; a left atrial appendage occlusion device with an endoskeleton, the endoskeleton constructed of a flexible material and comprising barbs, a barrier layer; and further performing the steps of inflating the balloon from a deflated form to the preformed shape; expanding the endoskeleton with the inflated balloon; plastically deforming the endoskeleton from a first, compressed form to a second, expanded form; deflating the balloon; removing the balloon and the delivery catheter; wherein the endoskeleton is configured to remain in the second, expanded form.

In another embodiment the endoskeleton further comprises barbs, and further comprising the step of pushing the barbs into the left atrial appendage.

In one embodiment the endoskeleton is comprised of a material with an elastic modulus between 100-310 GPA. In another the hypotube has a pattern cut into it, the pattern configured to allow the hypotube to expand outward under pressure from the balloon.

In one embodiment the further step of injecting contrast media to identify the presence of blood flow between the left atrium and the left atrial appendage is performed.

In another, the additional steps of inserting a sizing balloon into the left atrial appendage, inflating the sizing balloon with a known amount of inflation media, deflating the sizing balloon, removing the sizing balloon, and using the known amount of inflation media to calculate the size of the left atrial appendage are performed.

### BRIEF DESCRIPTION OF THE DRAWINGS

FIG. 1 is a partial perspective view of a left atrial appendage occlusion device;

FIG. 2 is a cross sectional view of the shaft of left atrial appendage occlusion device of FIG. 1 along the line A:

FIG. 3 is a cross sectional view of the balloon of FIG. 1 along the line B, inside a left atrial appendage:

FIG. 4 is a partial perspective view of a left atrial appendage occlusion device of the present invention:

FIG. 5 is a partial perspective view of a left atrial appendage occlusion device of the present invention:

FIGS. 6A-C are partial perspective views of a balloon system of the present invention:

FIGS. 7A-C are partial perspective views of an implant system of the present invention;

FIG. 8A is a partial perspective view of a balloon system of the present invention;

FIG. 8B is a partial perspective view of a balloon system of the present invention;

FIG. 8C is a partial perspective view of a balloon system of the present invention;

FIG. 9 is a 2D diagram of an endoskeleton pattern of the present invention;

FIG. 9A is a partial 2D diagram of an endoskeleton pattern of the present invention;

FIG. 9B is a 3D view of an endoskeleton pattern of the present invention;

FIG. 9C is a 3D view of an endoskeleton pattern of the present invention;

FIG. 9D is a 3D view of an endoskeleton pattern of the present invention;

FIG. 9E is a 3D view of an endoskeleton pattern of the present invention;

FIG. 10 is a partial perspective view of an implant system of the present invention in a deflated state;

FIG. 11 is a partial perspective view of an implant system of the present invention in an inflated state.

### DETAILED DESCRIPTION

The disclosure generally relates to methods and devices for occluding a left atrial appendage (“LAA”). Some aspects of the disclosure relate to implantable devices adapted, sized and configured for LAA occlusion. Some aspects of this disclosure can be used in the event of a catastrophic failure, such as a LAA rupture. Some aspects of the different embodiments herein, however, may be suitable for incorporation into different embodiments, including devices and methods.

The first part of this this disclosure generally describes devices adapted for and methods of treating a LAA rupture. A LAA rupture can be caused by a minimally invasive procedure in which an attempt is made to implant a LAA occlusion and/or closure device, such as, for example, the Watchman™ device sold by Boston Scientific. Methods herein may also include, following a rupture in the LAA, sealing a hole in the LAA. The methods herein can include temporarily occluding a LAA, but in some instances the method can include a permanent occlusion of the LAA.

During the implantation of some LAA occlusion and/or closure devices, an access sheath is already in place in the atrium to facilitate advancement of the delivery system. In the event of a LAA rupture (such as caused by a guidewire or the implantable device), the guidewire or delivery system are removed from the access sheath (depending on when in the procedure the rupture occurs), leaving it available for another device to be quickly inserted through its lumen and to the LAA. The presence of a previously-placed access sheath or guiding catheter (or other access device with a lumen therein) is generally an important step in this method, in that a subsequent procedure to treat the LAA rupture needs to be performed almost immediately after the rupture, and the existing access sheath or guiding catheter provides a device that facilitates delivering the necessary tools very quickly to the LAA.

After detecting a ruptured LAA, a separate occluding device can be quickly advanced through the previously placed access sheath. An expandable device, which may include an expandable membrane, can then be expanded against LAA tissue, LAA ostium tissue, or left atrium tissue adjacent a LAA ostium, in an attempt to occlude or isolate the LAA from blood flow from the left atrium. By preventing blood from the left atrium into the LAA, any leak in LAA tissue can be isolated from the left atrium to prevent further blood loss from the left atrium.

After an attempt has been made to occlude the LAA and isolate a leak from the left atrium, it can be determined if blood is flowing from within the LAA to a location outside the LAA. Using the occluding device, a dye can be injected into the LAA, followed by visualizing the dye to determine if the dye is moving from a location inside the LAA to outside the LAA, which indicates the existence of an opening (aka a leak) in the LAA. If an opening is visually detected, the method can further comprise sealing the opening. For example, without limitation, sealing an opening in the LAA comprises using the device to inject a sealant into the LAA to seal off the opening in the LAA. The device can include an aperture out of which the sealant is delivered into the LAA. The sealant, which can be a wide variety of biocompatible adhesives, will seal off the opening when exposed to the tissue with the leak. After attempting to seal the opening in the LAA, dye can then be injected once again into the LAA, followed by visualization to determine if the dye is moving from a location inside the LAA to a location outside of the LAA, which indicates the continued existence of an opening in the LAA. The step of injecting adhesive can be repeated as necessary, followed by dye injection and visualization, until the opening has been sealed off. Once the opening has been effectively sealed, the patient can then be monitored for any length of time as needed.

In some embodiments the device is temporary and is removed from the patient once it is determined that the opening is sealed. In some instances, however, the device is adapted to be left in place permanently as the implantable LAA occlusive and/or closure device.

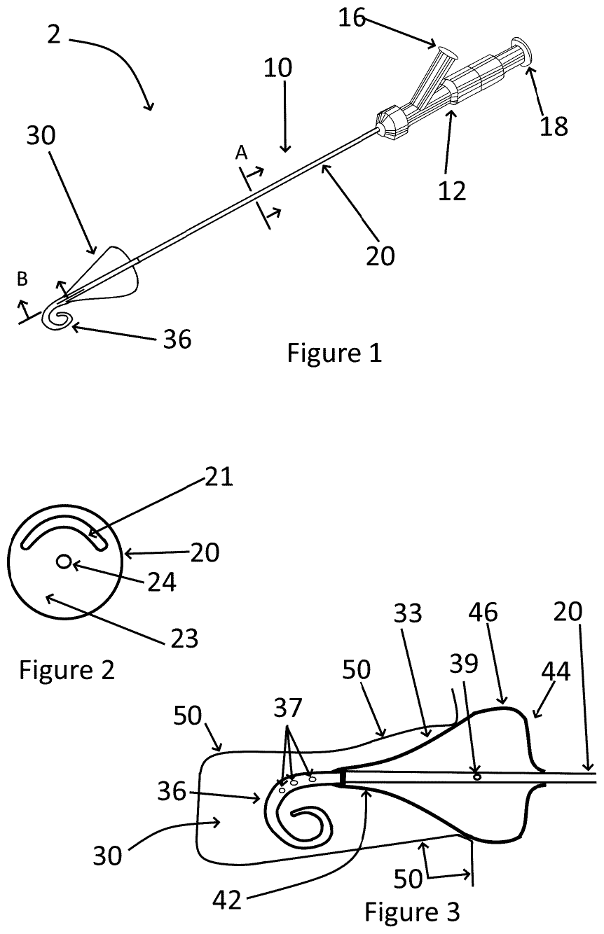

FIGS. 1-3 illustrate a left atrial appendage occlusion device 2 for occluding a left atrial appendage. Occlusion device 2 includes external portion 12 coupled to elongate member 10 that comprises shaft 20, and expandable member 30 carried by a distal region of shaft 20. Expandable member 30 is shown in an expanded (in this case inflated) configuration. However, during initial delivery to the LAA, the expandable member is in a deflated, or collapsed state and thus has a lower cross section that more closely approximates the cross section of the elongate shaft or a sheath (not shown). External portion 12 includes inflation fluid port 16 and internal lumen port 18.

As can be seen in the sectional view of FIG. 2 taken along section A-A shown in FIG. 1, inflation fluid port 16 is in fluid communication with inflation lumen 21, and internal lumen port 18 is in communication with main lumen 23. Main lumen 23 may be the space between the OD of an internal catheter or hypotube, and the ID of an external catheter's tubing. In one embodiment the inflation lumen is flexible, such that its volume may rise or fall depending on the pressure of the inflation material. As such, the inflation lumen may expand and take up more space during inflation of the expandable member, but collapse and take up less space during the remainder of the procedure. Likewise, additional lumens may be present. For example, there may be a lumen for injecting contrast media (not shown). There may be multiple contrast lumens to correspond with multiple contrast ports. There may also be one or more adhesive lumens 24, corresponding with one or more adhesive ports (not shown), for applying an adhesive to the LAA to seal it. In particular, once the location of the leak is determined, a preferred adhesive port may be identified for applying adhesive to the LAA to seal the leak. There may also be a guidewire lumen, or central lumen 24 may serve as the guidewire lumen.

LAA occlusion device 2 includes an expandable member 30 (in this embodiment including an expandable membrane 33, or balloon) carried by shaft 20 of elongate member 10, either by being directly attached to catheter 10 or indirectly attached to catheter 10. Distal to expandable member 30 is distal elongate member 36, which includes at least one aperture 37 therein (three apertures 37 are shown).

Elongate member 10 includes a shaft 20 and has a first lumen 21 therein in fluid communication with port 16 and inflation port 39, which is disposed in fluid communication with (in one example, inside) expandable member 30. An inflation fluid can thus be delivered into port 16, down lumen 21 and out port 39 and into expandable member 30, causing the expansion (in this case inflation) of expandable member 30.

In this embodiment expandable member 30 includes a membrane 33 that has an inflated configuration configured to close off the LAA after it has been inflated. The shape is important in that the goal of inflation is to isolate any holes in the LAA from the left atrium. The membrane 33 thus optimally will seal off the LAA from the left atrium.

In this embodiment the membrane 33 has a general pear shape, with the greatest height (see Height “H” dimension shown in FIG. 3; orthogonal to longitudinal axis “LA”) in a proximal region 46 of the membrane 33. When a mid-point of the length of the expandable member 30 is determined, proximal region 46 is, in this embodiment, proximal to the mid-point. The membrane tapers downward from the greatest height dimension in the distal direction. Since there is patient-to-patient variability in the shape of the LAA, the membrane's pear configuration allows it to be securely engaged against LAA or ostia regardless of the specific patient configuration and size. The membrane, once expanded, can be advanced as far distally as it needs to be in order to engage tissue and isolate the LAA. The isolation step is critical to stabilize the blood leakage out of the heart. Membrane 33 has a configuration that allows it to be expanded so as to snugly fit against the LAA or at the location of the ostia, and isolate the LAA.

In one method, the pear-shaped membrane may be expanded before entering the LAA, or it may be expanded after it partially enters the LAA. In these two embodiments the membrane is then pushed further into the LAA to securely or snuggly fit around the entire perimeter of the LAA, closing it off to blood flow. In another embodiment, the membrane may be fully maneuvered into the LAA or the LAA opening before expansion. Expansion then pushes the membrane into the LAA or the opening to close off the blood flow. Finally, in another embodiment the membrane may be pushed past the LAA opening and fully into the LAA, where it is then expanded and pulled back proximally to seal the LAA.

While the membrane is depicted in FIG. 1 as being pear shaped, the membrane may also be cylindrical, hour glass shaped, conical, spherical, or ball shaped. Each of these membrane styles may be used with each of the methods in the above paragraph, and use the other elements depicted in FIGS. 1-3.

In preferred embodiments (but not limiting), the membrane is manufactured to have a particular configuration at maximum volume. That is, when the membrane is inflated with a known volume of fluid (air or liquid), the membrane will assume the pre-set, or manufactured geometry. This allows the membrane to always assume the desired configuration when inflated inside the patient's LAA or ostium. The membrane can comprise a silicone material, such as the balloon of a Foley Catheter, whose constructions are known. The membrane can also be polyurethane or nylon. The material of the membrane is generally very thin, and is meant to be a compliant material. Preferred materials have a Shore A hardness of 70-90A.

In the embodiment depicted in FIG. 1, the expanded configuration of membrane 33 includes a proximal region 46 with a greater height than distal region 42, with the membrane tapering radially inward in the distal direction relative to proximal region 46. The expanded configuration also includes a proximal most region 44 that extends radially outward, relative to longitudinal axis LA, from the proximal end of the membrane to greatest height region 46.

The membrane is volumetric, that is, it is inflated with a known volume of fluid such that the membrane assumes the known configuration, and is not pressure related. The membrane can be inflated at relatively low pressure.

The material of the membrane can be elastic, or in some embodiments it may be generally inelastic and have a pre-set configuration with inelastic material.

The distal member 36 is distal to expandable member 30 and is atraumatic. In the embodiment shown in FIGS. 1-3 the distal member has a pig-tail configuration, which will naturally be able to find the end/bottom of the LAA atraumatically. The distal member can be made from any number of flexible materials, such as a variety of polymeric materials, and may form any atraumatic shape.

Distal member 36 can be secured to elongate member 10 and/or expandable member 30 using any known securing techniques, such as adhesive bonding. Distal member 36 may also be an extension of shaft 20, though in this case it may need to be a lower durometer or less stiff material.

The occlusion device 2 also includes an inflation port 39 in the shaft 20, within membrane 33, that allows an inflation fluid to be delivered out of port 39, at a known volume, so membrane 33 will assume the pre-set configuration when inflated with the pre-set volume.

In one method of use, device 2 is advanced distally through a previously positioned access sheath or guiding catheter (not shown). Expandable member 30 is advanced out of a distal end of the access sheath and into the left atrium of the LAA. At this time the expandable member 30 is in an unexpanded (in this case uninflated) configuration. The device is continued to be advanced until distal member 36 is disposed within the LAA. In one approach the device is advanced until resistance is felt. A predetermined volume of inflation fluid is then advanced, from port 16, down inflation lumen 21, out aperture 39, and into expandable member 30 to cause membrane 33 to assume the known configuration, as is shown in FIG. 3. Inflation of the membrane causes the expandable member 30 to engage tissue at locations 50, sealing off the LAA from the left atrium. The expandable member may alternatively be inflated to max volume and then advanced distally into the LAA until the distal member engages LAA tissue.

A dye/contrast is then injected through a port, e.g., 16, 18, or another port, through a lumen, e.g., 21, 23, 24, or another lumen, and out one of apertures 37. The dye can be visualized under fluoroscopy to determine if it a leak exists in the LAA. If a leak is detected, a sealant is then delivered from outside the device, through the device in one of the lumens, e.g., lumen 23 or a third lumen (not shown), and out one of apertures 37. The sealant will seal off the detected leak. Dye is then injected through the device again and out one of apertures 37 to determine if the dye leaks out of the LAA. The sealing and dye procedure is repeated until it is determined that the leak has been sealed off and dye is no longer leaking out of the LAA. The dye and sealant can be delivered through different lumens, or the same lumen, and can likewise exit through the same or different apertures 37.

Once the leak is sealed, the device can be removed from the patient. In some embodiments, however, the device can be adapted to be a permanent implant and is adapted to be detached from a system. Likewise, after one or more of the steps listed above, the following embodiments described herein may be used to provide an LAA occluding implant in the LAA.

For example, while not all of the steps may be followed in every case, in one method one of the inflatable members described above is delivered to one of the LAA locations described above. It is inflated against the LAA tissue, LAA ostium tissue, or adjacent the LAA ostium. It is then inflated, and a dye is injected to determine if the dye is flowing between the LAA and outside the LAA. If dye is still flowing, the inflatable member may be deflated, moved, and reinflated. The above steps could then be repeated as needed, until the flow is stopped. Alternatively, the inflatable member could be further inflated, and again the above steps could then be repeated as needed, until the flow is stopped. Alternatively, or in conjunction with either or both of the above, if a leak is detected, a sealant is then delivered from outside the device, through the device in one of the lumens, e.g., lumen 23 or a third lumen (not shown), and out one of apertures 37. The sealant will seal off the detected leak. If a leak is not detected, or if the physician determines the sealant is not necessary, this step is optional. Subsequently, the inflatable member may be withdrawn and replaced by an expandable LAA occluding implant as described below, or the inflatable member may be secured in place and serve as an implant itself. In this case it may be useful to have tines or securing members on the expandable member. In one embodiment the procedure is as above, but the occluding implant is secured before the sealant is deployed for the leak.

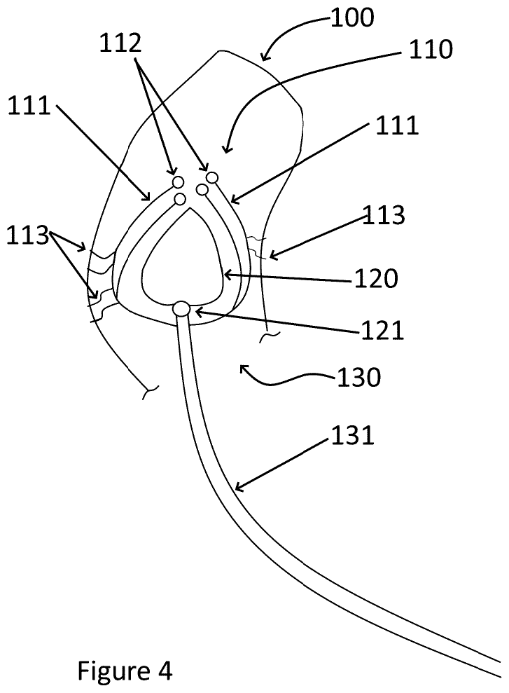

FIG. 4 illustrates a distal portion of an implantable occlusion device 110 that can be used to seal off the LAA 100 from blood flow from the left atrium, optionally in the case of an emergency situation as described above. Alternatively, device 110 can also be used in non-emergency situations, and can be used as a permanent LAA occlusion implant, rather than just temporarily placed. The delivery access routes and procedures described above can similarly apply to this embodiment as well.

The occlusion device 110 includes a plurality of expandable elongate members 111 that optionally include atraumatic distal ends 112 (only one atraumatic distal end is labeled for clarity). The expandable elongate members 111 can be in the form of elongate splines, or arms, and alternatively they could be overlapping and/or interwoven elongate members, such as a braid. Expandable elongate members 111 can be made from an elastic material, such as nitinol. In this embodiment, the elongate members 111 are not coupled to one another, or to anything else, at their distal ends (i.e., they have free distal ends, or open distal ends), and they together define an open volume radially within the elongate members 111. The elongate members can be thought of as defining an open-ended cage. In one embodiment the elongate members will be coupled with a covering as described below, and together form an implant. In other embodiments the elongate members are themselves the implant.

The elongate members are not directly coupled to anything at their distal ends. In one embodiment the elongate members are also not coupled directly to anything along most of their lengths. In some embodiments the elongate members 111 are not directly coupled to anything along at least 50% of the lengths (starting at their distal ends), along at least 55% of the lengths, along at least 60% of their lengths, along at least 75% of the lengths, or more (e.g., 80%, 85%, 90%, 95% of their lengths). “Directly coupled” in this context includes branching elements that branch from the elongate member (to form more than one elongate member). So, when the elongate members are described as not being directly coupled to anything, that includes that the elongate members don't having any branching members that extend from the elongate member. Another way of describing that is the elongate member is a single elongate member from which a branching element does not extend, and the elongate member is not directly coupled, or attached, to another component in its distal portion.

In any of the embodiments herein, there can be two, three, four, five, six, seven, eight, nine, ten, or more expandable elongate members. There can be as many as can be included based on any particular design. In some embodiments there are from 2 and 30 expandable elongate members 111. In other embodiments there are 20-50 expandable elongate members 111.

Each of the expandable elongate members 111, when expanded outward as shown, have a configuration that bows outward relative to an axis of shaft 131, then after reaching a max radially outward dimension, extends back radially inward toward the longitudinal axis of shaft 131, as shown. The height changes more quickly in the proximal portion of the device, and reduces more gradually on the distal side of the max height dimension, as shown.

At least one of the elongate members 111 carries at least one hook, barb, or other type of piercing element 113 (only 2 are labeled in FIG. 4) adapted to pierce LAA tissue and anchor the occlusion device relative to the tissue. The hooks or barbs can be integrally formed with any of the elongate members (i.e., made from the same starting material), and can be adapted to expand or change position relative to the elongate member upon release from a delivery device (e.g., expand further radially outward). The hooks can be the same material as the elongate members. They can also have different properties as the elongate materials, such as different thicknesses. They can be less flexible than the elongate members 111, for example.

Occlusion device 110 is adapted to be expanded with an inflatable balloon 120, rather than being self-expanding. Balloon 120 has a preformed shape (such as with a generally inelastic material) when inflated with a known volume. The balloon can be molded to have a shape that resembles typical LAAs. Balloon 120 may also be compliant, that is it can easily conform to varying LAA anatomy or size. Balloon 120 has a generally tapering configuration, and can be generally cone or pear shaped. In some embodiments, the balloon is made of polyurethane, nylon, or silicone, and is relatively thin. Balloon 120 can be carried by the distal end 130 of delivery device 110, such as a delivery cable or shaft, and can be in fluid communication via a fluid lumen with a fluid source external to the patient, such as is described above with respect to FIGS. 1-3. The sealing steps and features from FIGS. 1-3 are incorporated into the FIG. 4 embodiment as an additional safety net during the procedure as needed.

Thus, while not all of the steps may be followed in every case, in one method one of the occlusion device 110 described above is delivered to one of the LAA locations described above. The balloon 120 is inflated against the LAA tissue, LAA ostium tissue, or adjacent the LAA ostium. A dye is injected to determine if the dye is flowing between the LAA and outside the LAA. If dye is still flowing, the balloon 120 may be further inflated, or may be deflated, moved, and reinflated. The above steps could then be repeated as needed, until the flow is stopped. Alternatively, the inflatable member could be further inflated, and again the above steps could then be repeated as needed, until the flow is stopped. Alternatively, or in conjunction with either or both of the above, if a leak is detected, a sealant is then delivered from outside the device, through the device in one of the lumens, e.g., lumen 23 or a third lumen (not shown), and out one of apertures 37. The sealant will seal off the detected leak. If a leak is not detected, or if the physician determines the sealant is not necessary, this step is optional. Subsequently, the balloon 120 may be withdrawn and replaced by an expandable LAA occluding implant as described below, or the balloon 120 may be secured in place and serve as an implant itself. In this case it may be useful to have tines or securing members on the expandable member. In one embodiment the procedure is as above, but the occluding implant is secured before the sealant is deployed for the leak.

Once the device 110 and uninflated balloon are delivered to the above locations through the existing sheath, the balloon is inflated, which pushes the expandable members 111 radially outward. In this embodiment, the plurality of elongate members are not self-expandable, but rather are balloon expandable. In other embodiments, they could be self-expandable or partially self-expandable, and further expanded with balloon 120. When expandable members 111 are urged radially outward, hooks 113 pierce through LAA tissue, securing the device 110 with respect to LAA tissue.

The balloon 120 can be inflated until the outermost dimension (relative to a longitudinal axis) of the occlusion device 110 has reached a desired size. In one embodiment the device 110 does not have a premade, expanded configuration, like a self-expanding device. It assumes a shape in situ when expanded by balloon 120. This allows the device to be a one-size fits all device, and it is expandable until it reaches the desired size, which can be confirmed by ensuring blood is not entering into the LAA from the left atrium, using any of techniques described herein.

Generally, the device 110 is expanded until blood is not entering from the atrium into the LAA, and methods for assessing the same are described above and applicable in this embodiment.

In one embodiment, once the device 110 has sufficiently occluded the LAA, the balloon 120 is deflated and removed through a central region 121 of occlusion device 110.

The central region 121 can be thought of as a trap door, and may function similar to a one-way valve through a central region of the device 110.

The plurality of elongate members 111 are secured to a material that extends between the elongate members and acts to occlude blood. The material extends across at least a proximal portion of the expandable device 110.

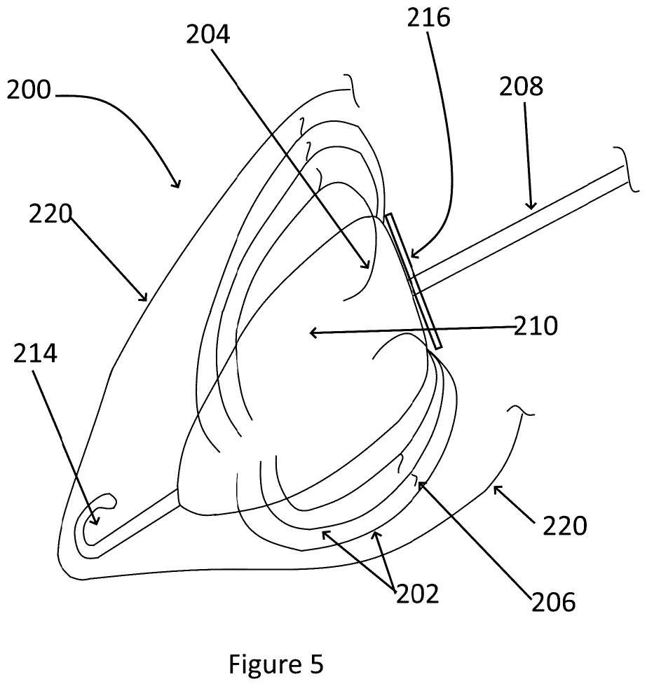

FIG. 5 illustrates another exemplary embodiment that is similar to the device shown in FIG. 4, and any suitable disclosure for FIG. 5 can be incorporated by reference with the example in FIG. 5 unless specifically indicated to the contrary. FIG. 5 illustrates an implantable LAA occlusion device 200, after it has been delivered to a position within a LAA. The implantable device 200 includes a plurality of elongate elements, 202, which are similar to members 111 from FIG. 4. In this embodiment, however proximal end regions of the elongate elements include a folded, or bent, region 204. They can be bent backwards towards the distal end to some degree prior to expansion, or can be extending generally radially inward toward the longitudinal axis of the device prior to expansion. Each elongate member has an atraumatic distal end, such as a ball, curled region 214, etc., which minimizes damage to LAA tissue. The ends could even have, for example, pigtail configurations. The elongate elements 202 can include anchors 206 that can be in form of hooks or barbs, as did elongate members 11 from the example in FIG. 4. All of the disclosure regarding hooks or barbs from FIG. 4 can apply to anchors 206. Each of the elongate members 202 can have more than one anchor 206.

Device 200 is, like the device in FIG. 4, open ended at its distal end.

The device in this embodiment can be delivered using a delivery and expansion device, which includes elongate shaft 208, which can have a flexible distal region with a pigtail configuration, but which can be straightened if being delivered “over the wire.” The distal end of shaft 208 can also have one or more ports therein, which can be used as injections ports to assist with determining if there is a leakage between LAA and the atrium, similar to ports 37, 39 from FIG. 3. The delivery device also includes balloon 210 mounted to shaft 208, which is pre-shaped and can function like balloon 120 from FIG. 5. The balloon is in fluid communication with a fluid lumen within elongate shaft 208.

An exemplary “over the wire” placement of the implantable device now follows. LAA wall 220 is shown in FIG. 5. If the access is transeptal, a wire can be placed in the LAA, and the septal sheath can still be in position extending across the septum. The implantable device, while secured to the placement and expansion device, can then be advanced over the wire that has already been placed. The wire can be removed, allowing the pigtail 212 to form an atraumatic distal end. The balloon can then be inflated as described above for FIG. 5, and can continued to be inflated until it is believed that the elongate member and optional anchors are anchored to tissue. The barrier 216 secured to the proximal end regions of the elongate elements can begin to assume a larger configuration due to the elongate elements expanding. Optionally, the proximal regions of the elongate members that are bent backwards can be designed so that as the device is expanded, the proximal end regions deform and begin to extend more directly radially inward, together forming a somewhat flattened proximal end of the implant, across which the barrier extends. The expansion process can thus allow the proximal ends to deform and create a proximal end of the expandable portion of the implant device.

Elongate elements 202 and bent back regions 204 may be free, and unattached. Alternatively, they may be attached to a balloon 210. They may be attached or woven into a barrier 216, e.g., a knit material. They may also be attached to a ring (not shown). The attachment point can be at the junction between the elongate elements 202 and the back region 204, or may be at multiple points.

The shaft 208 can have ports distal to the balloon 210 that can be used to inject dye to check if fluid is leaking from within the LAA to a location in the atrium. If it is, the balloon can be inflated more, and leakage can then be rechecked. The process can be continued until no leakage is detected, and the device is determined to have been expanded sufficiently.

The balloon 210 can then be deflated, and removed through a central opening in the proximal region of the implantable device. The bent back regions 204 can be adapted so that when the balloon is deflated, the bent back regions 204 can also revert so that they are extending in a more radially inward configuration, helping create the barrier configuration of the proximal end of the implantable device. Optionally still, the proximal bent back regions 204 of the elongate members 202 can be adapted so that as the balloon and delivery device 208 are retracted proximally, the bent back regions 204 can continue to deform so they extend radially inward. In still further embodiments, the proximal regions 204 are adapted to interact with one another as they revert to the different configuration, and can lock together or at least become more stabilized relative to one another.

Any of the elongate members in FIGS. 4 and 5, even though they may have a curved configuration in a side view of the elongate member from a proximal end to a distal end, can also have linear configurations when viewed 90 degrees offset in a top or bottom view.

While some aspects of this disclosure are related to a one-size-fits-all type of implantation occlusion device, an additional aspect of this disclosure is related to more effective methods and devices for selecting a proper size or dimension (e.g., diameter) of an LAA occluding implant, such as any of the implants herein or other implants such as a Watchman™ device (FDA approved in 2015). An exemplary advantage of the methods and devices in this aspect is that an implant may be selected from a plurality of differently-sized implants for implantation that more closely approximates the size of the patient's LAA. An additional exemplary but optional advantage of the methods and devices herein is that fewer different implant sizes may need to be available for implantation to prep for the procedure if a more accurate determination or estimation of the size (at least one dimension) of the patient's LAA is made. For example, it may be possible to only need two or three differently sized implants (for example only) to be available for the implantation procedure rather than five or more differently sized implants commonly required by prior art devices.

Existing approaches to estimate a patient's LAA size may include using imaging techniques (such as CT or ultrasound, e.g., TEE) to generate one or more images of one or more parts of the LAA and/or LA. The images may be utilized (e.g., manually visualizing the images) to estimate a LAA size, and then an implant size may be selected from a variety of possible implant sizes based on the estimation.

The disclosure in this aspect includes a method of estimating a size of a patient's LAA and selecting an occlusion implant to be implanted from differently-sized implants. The method includes expanding an expandable member (optionally an inflatable membrane) within an LAA, ostium and/or left atrium tissue adjacent a LAA ostium, and determining when at least a portion of the expandable member approximates at least a portion of the size of the LAA (e.g., engages tissue). Once at least a portion of the expandable member has been determined to approximate a size of at least a portion of the LAA (or at least approximates it closely enough), the method may include determining and selecting an implant size for implantation based on one or more of 1) the size of the expandable member when it approximates the size of the LAA and/or 2) at least one aspect of the expansion of the expandable member (e.g., volume of fluid delivered to the expandable member). Selecting an implant may include selecting an implant from a plurality of possible implants, each having a different size (i.e., at least one different dimension, such as diameter).

In this aspect, the expandable sizing member may include an inflatable and deflatable balloon, such as is shown in FIGS. 1-3, above, and described in paragraphs 44-62, above. A sizing balloon in a mostly or completely deflated (not expanded) state or

configuration may be advanced on a delivery device (e.g., including an outer shaft coupled to the proximal end of the balloon, and a smaller axially movable inner shaft coupled to a distal end of the balloon). An inflation lumen may be defined between the outer shaft and the inner shaft such that inflation fluid may be delivered distally through the inflation lumen and into the volume inside the balloon to inflate the balloon, as shown. Alternatively, or additionally, the inflation fluid may be delivered through the inner shaft, and the inner shaft may have one or more openings therein out of which the inflation fluid (a gas (e.g., air), liquid (e.g., saline), etc.) passes into the balloon to inflate the balloon. Additionally still, the inner shaft may be axially withdrawn relative to the outer shaft to cause at least partial balloon expansion away from the axis of the shaft.

In use, the inflatable sizing balloon may be expanded at least partially within the LA, the ostium and/or the LA (e.g., it may be expanded and then pushed distally against the ostium). The sizing methods herein may include determining if and when at least a portion of the expandable member (e.g., greatest diameter portion), after at least partial expansion, engages tissue and approximates the size of at least a portion of the LAA, which can provide an indication that the particular state or configuration of the expandable sizing member is indicative of (or can be correlated to) an implant size to be selected for implantation to accurately fit the patient's LAA.

In some instances, determining if and when at least a portion of the expandable member (e.g., greatest diameter portion) approximates the size of at least a portion of the LAA can include determining if the expandable member is engaging (or mostly engaging) LAA and/or ostium tissue. One method of determining if the expandable member is engaging tissue is delivering a dye into the LA through the system and determining if at least some of the dye passes from the LA and into the LAA, which would suggest the expandable sizing member is not, at least to some extent, fully engaging or pushed against tissue. It may, however, be possible to determine that the expandable member has assumed a state or configuration that approximates the LAA size closely enough even if there is some minimal amount of dye in the LAA. Alternatively stated, it may be possible to accurately determine an appropriate implant size even if there is some dye in the LAA. Determining if the dye flows in the LAA may be determined using imaging techniques, such as radiographic imaging techniques.

The method can also include, after determining that at least a portion of the expandable member approximates the size of the LAA, selecting an implant size for implantation based on one or more of 1) the size, state or configuration of the expandable member when it approximates the size of the LAA and/or 2) at least one aspect of the expansion of the expandable member (e.g., total volume of fluid delivered to the expandable member). For example, a volume of inflation fluid (e.g., saline) delivered into the expandable sizing member may be tracked or monitored, and a known (pre-existing) correlation or relationship between volume delivered and diameter of the expanded balloon can be used to determine the diameter of the balloon in the patient after a certain volume of fluid is delivered. In this example, the diameter of the balloon in the patient is known based on the total volume delivered, and thus the diameter of the balloon can be easily determined when the sizing member most closely approximates the size of the LAA (or at any time during the balloon expansion). Using the balloon diameter to “size” the LAA in this manner, the implant size can then be selected so that the implant will more accurately fit the patient's LAA size. It is of course contemplated that other methods of correlating expansion of the balloon to the diameter of the balloon during the expansion (and thus approximation) can of course be utilized.

In some methods, the expandable sizing member may be incrementally expanded (e.g., incrementally inflated), and periodic checks on tissue approximation may be performed to determine when the expandable member has been expanded sufficiently to allow an accurate implant size to be determined. For example, a first known volume of fluid (e.g., 1 cc) may be delivered to inflate the balloon, followed by checking to see if the sizing member sufficiently engages tissues (e.g., with a dye shot/check). If too much dye enters the LAA, for example, a known volume of fluid (e.g., 1 more cc) may be delivered to further expand the sizing member (e.g., to 2 cc total), followed by again checking to see if the sizing member sufficiently engages tissues (e.g., with a dye shot/check). This process can be performed incrementally until it is determined (e.g., by a physician and/or using an algorithm) that the sizing member is adequately expanded, and the implant size can then be selected based on the known relationship between volume delivered and diameter of the sizing member.

Alternatively, the balloon may be further expanded to a greater extent without adding any fluid. For example, an inner shaft may be pulled proximally relative to the outer shaft, bringing the distal end of the balloon towards the proximal end. This may increase pressure in the balloon, possibly causing further expansion (greater diameter). Expansion of a balloon may thus be hydraulically/pneumatically driven and/or mechanically driven. Axial movement(s) of the inner shaft (which in some embodiments may be monitored externally in a handle, for example) can similarly be correlated with increases in diameter of the balloon, for example (e.g., known axially movement associated with a known increase diameter).

The sizing member (e.g., sizing balloon) can then be collapsed (e.g., deflated) and removed proximally through the sheath, with the sheath maintained in the LA. In this aspect, once the implant size is determined or selected, the selected implant can then be delivered through the sheath in a collapsed state, expanded, and implanted in the LAA, examples of which are shown herein in FIGS. 4 and 5. The implant may also have a variety of known implant configurations, and may be similar to the Watchman™, for example without limitation.

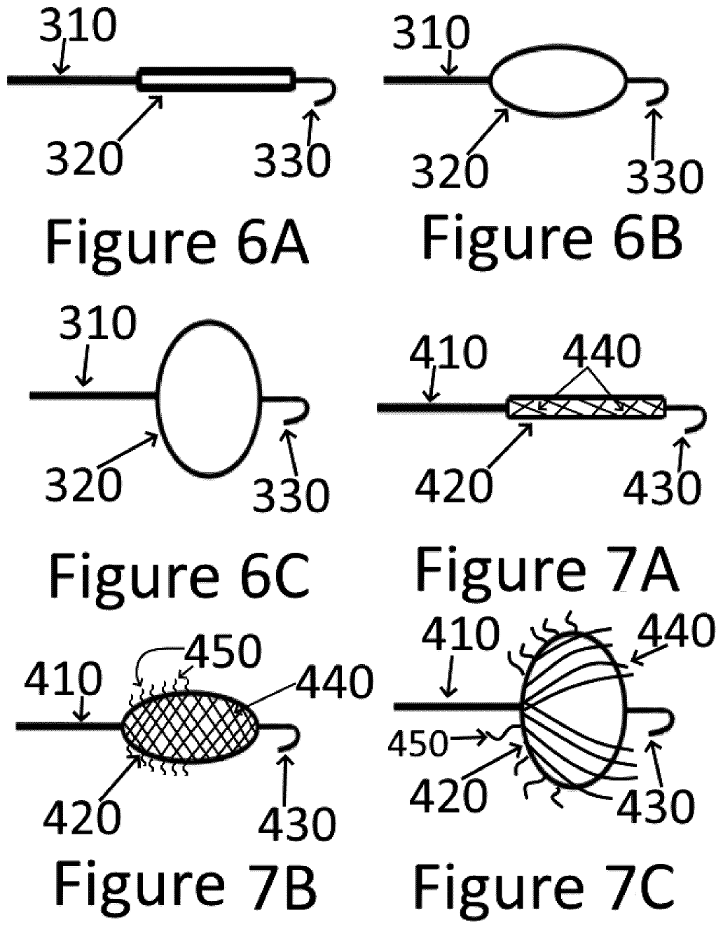

FIGS. 6A-C illustrate delivery and expansion of an expandable sizing member 300 (in this case a sizing balloon). Sizing member 300 includes a shaft 310, a balloon 320, and an atraumatic tip 330. FIG. 6A shows a sizing balloon 320 deflated for delivery. FIG. 6B shows a sizing balloon 320 partially inflated. FIG. 6C shows the balloon 320 fully inflated. The sizing balloon can be deflated and removed in the reverse order (right to left in FIGS. 6A-C).

FIGS. 7A-C illustrates a balloon expandable LAA occlusion device 400 delivered on a balloon 420 and shaft 410. FIG. 7A shows the device in a collapsed state. Thus, elongate elements 440 are flat on the balloon 420. FIG. 7B shows the device 400 with the balloon partially expanded. Elongate elements 440 are at least partially expanded, and anchors 450 are expanded. FIG. 7C shows the device fully expanded (e.g., by proximally retracting an inner sheath to which the distal end of the balloon is coupled). The balloon may then be deflated and removed, leaving the implant in place. The delivery system may then be removed.

In some embodiments, the implant may be delivered through the sheath and into the LA in a collapsed state about the expandable member (e.g., inflatable balloon) such as shown in FIG. 7B. The implant is delivered into the LAA and expanded by inflating the balloon, additional examples of which are described herein.

In one embodiment, the LAA occlusion device consists of four systems, a sizing system, a delivery system, a balloon system, and an implant system. The systems can of course be combined into one system, two systems, etc.

The procedure would typically begin with accessing the left atrium through a typical transseptal access. In one common approach a long sheath with a dilator is introduced through the femoral vein and advanced into the superior vena cava over a guide wire. The fossa ovalis is crossed via a BRK needle. The needle is removed and a guidewire, e.g., a 0.035″ diameter guidewire, is left in the left atrium. The delivery system consists of a dilator and a curved or steerable sheath. The sheath may be precurved, may be steerable via pull wires, magnetic guidance, or have a steering element that may be inserted to direct the delivery system to the fossa ovalis. The dilator passes through the fossa ovalis with the sheath, and is removed. The guidewire is advanced into the LAA. At this point the delivery system is in place.

While an exemplary delivery system is discussed above, other delivery systems and other approaches than a femoral entry are known in the art are appropriate for use herein.

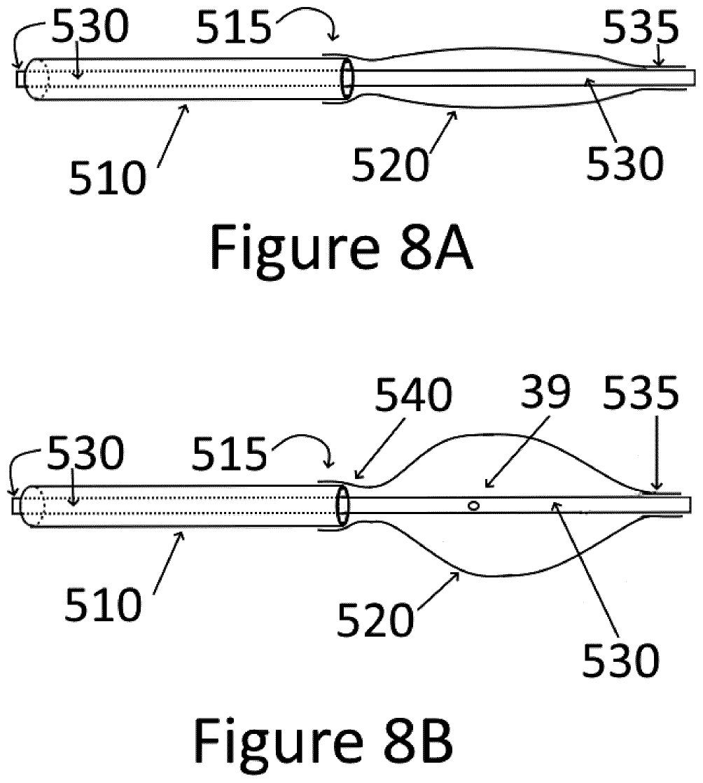

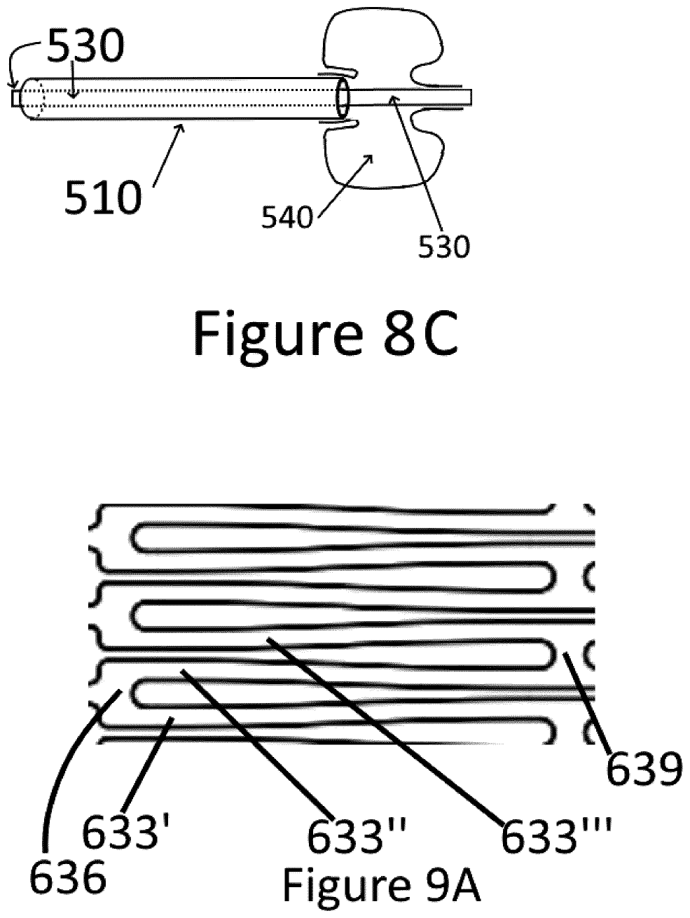

The sizing system is discussed above, and consists of a sizing balloon catheter, a sizing balloon. The sizing system is depicted in FIGS. 8A-8C, and consists of balloon outer catheter 510. The proximal end of balloon 520 is attached to balloon outer catheter 510 at or near the distal end 515 of the balloon outer catheter. Inner balloon catheter 530 extends throughout a lumen of catheter 510, past the distal end of outer balloon catheter 510. The distal end of balloon 520 attaches to the distal end 535 of inner balloon catheter 530. Inner balloon catheter 530 can be secured to outer balloon catheter 510 so that the length of the balloon is fixed. Alternatively, the inner catheter may be secured outside the patient via a Tuohy Borst valve or other mechanism (not shown). If the length of the balloon is to be adjusted as shown in FIG. 8C, the valve is opened and the catheters may be moved axially relative to each other, changing the length of the balloon.

As shown in FIG. 8A, the balloon is initially delivered in a deflated state. The balloon may be stretchable, and as such in its deflated state it conforms closely to the catheter. The balloon 520 may also be folded or otherwise compacted to reduce its cross section as much as possible for delivery through the heart to the LAA.

In an alternative embodiment, only one balloon catheter is provided, and both the proximal and distal ends of the balloon 520 are attached to it. In this embodiment the balloon 520's length may be fixed, or may be adjusted by bending (and thus shortening) the distal portion of the catheter 510. In another embodiment the balloon is attached to one or more rings that are axially slidable on the catheter 510. If the balloon is inflated beyond a certain point the ring may slide closer to the other end of the balloon, shortening the balloon (not shown).

In usage, the balloon assembly is delivered through the delivery sheath, above, to the LAA. An inflation lumen 540 may be defined between the outer shaft and the inner shaft such that inflation fluid may be delivered distally through the inflation lumen and into the volume inside the balloon to inflate the balloon, as shown in FIG. 8B. Alternatively as shown in FIG. 8B, or additionally, the inflation fluid may be delivered through the inner shaft, and the inner shaft may have one or more openings 39 therein out of which the inflation fluid (a gas (e.g., air), liquid (e.g., saline), etc.) passes into the balloon to inflate the balloon. Additionally, still, the inner shaft may be axially withdrawn relative to the outer shaft to cause at least partial balloon expansion away from the axis of the shaft as shown in FIG. 8C.

The inflatable sizing balloon 520 may be expanded at least partially within the LA, the ostium and/or the LA (e.g., it may be expanded and then pushed distally against the ostium). The sizing methods herein may include determining if and when at least a portion of the expandable member (e.g., greatest diameter portion), after at least partial expansion, engages tissue and approximates the size of at least a portion of the LAA, which can provide an indication that the particular state or configuration of the expandable sizing member is indicative of (or can be correlated to) an implant size to be selected for implantation to accurately fit the patient's LAA.

In some instances, determining if and when at least a portion of the expandable member (e.g., greatest diameter portion) approximates the size of at least a portion of the LAA can include determining if the expandable member is engaging (or mostly engaging) LAA and/or ostium tissue. One method of determining if the expandable member is engaging tissue is delivering a dye into the LA through the system and determining if at least some of the dye passes from the LA and into the LAA, which would suggest the expandable sizing member is not, at least to some extent, fully engaging or pushed against tissue. It may, however, be possible to determine that the expandable member has assumed a state or configuration that approximates the LAA size closely enough even if there is some minimal amount of dye in the LAA. Alternatively stated, it may be possible to accurately determine an appropriate implant size even if there is some dye in the LAA. Determining if the dye flows in the LAA may be determined using imaging techniques, such as radiographic imaging techniques.

The method can also include, after determining that at least a portion of the expandable member approximates the size of the LAA, selecting an implant size for implantation based on one or more of 1) the size, state or configuration of the expandable member when it approximates the size of the LAA and/or 2) at least one aspect of the expansion of the expandable member (e.g., total volume of fluid delivered to the expandable member, or distance between the distal and proximal ends of the balloon, or both). For example, a volume of inflation fluid (e.g., saline) delivered into the expandable sizing member may be tracked or monitored, and a known (pre-existing) correlation or relationship between volume delivered and diameter of the expanded balloon can be used to determine the diameter of the balloon in the patient after a certain volume of fluid is delivered. In this example, the diameter of the balloon in the patient is known based on the total volume delivered, and thus the diameter of the balloon can be easily determined when the sizing member most closely approximates the size of the LAA (or at any time during the balloon expansion). Using the balloon diameter to “size” the LAA in this manner, the implant size can then be selected so that the implant will more accurately fit the patient's LAA size. It is of course contemplated that other methods of correlating expansion of the balloon to the diameter of the balloon during the expansion (and thus approximation) can of course be utilized.

In some methods, the expandable sizing member may be incrementally expanded (e.g., incrementally inflated), and periodic checks on tissue approximation may be performed to determine when the expandable member has been expanded sufficiently to allow an accurate implant size to be determined. For example, a first known volume of fluid (e.g., 1 cc) may be delivered through port 39 to inflate the balloon, followed by checking to see if the sizing member sufficiently engages tissues (e.g., with a dye shot/check). If too much dye enters the LAA, for example, a known volume of fluid (e.g., 1 more cc) may be delivered to further expand the sizing member (e.g., to 2 cc total), followed by again checking to see if the sizing member sufficiently engages tissues (e.g., with a dye shot/check). This process can be performed incrementally until it is determined (e.g., by a physician and/or using an algorithm) that the sizing member is adequately expanded, and the implant size can then be selected based on the known relationship between volume delivered and diameter of the sizing member.

Imaging technologies such as transesophageal echocardiogram (TEE) and/or Intracardiac echocardiography (ICE) can be used to determine if the balloon is fully occluding the LAA ostium. Additional fluid volume is added to the sizing balloon until the TEE or ICE imaging confirms full closure of the LAA ostium. The volume of fluid used in the sizing balloon is recorded for use in the implant balloon catheter.

The sizing balloon 540 can then be collapsed (e.g., deflated and/or stretched back out axially) and removed proximally through the sheath, with the sheath maintained in the LA. In this aspect, once the implant size is determined or selected, the selected implant can then be delivered through the sheath in a collapsed state, expanded, and implanted in the LAA.

Once the sizing system has determined the proper size for the implant, the balloon system and implant system are used together to install the implant in the LAA. In one embodiment the implant balloon and system are similar to or the same as the sizing balloon system. Thus, the discussion above for the sizing balloon catheter 510 and sizing balloon 540 in FIGS. 8A-C also describe the implant balloon system. While the two systems may not be identical, it is contemplated that similar balloon systems will allow for a simplified algorithm for determining sizing, and for determining how far to inflate the implant balloon to properly seal the LAA with the implant system. Thus, while the sizing system may utilize an inner and an outer catheter, along with a balloon configured to allow a change in axial dimension as described above, the implant balloon system may only utilize one catheter (or vice versa).

In one embodiment, the implant system includes an endoskeleton and a barrier layer. In this embodiment the endoskeleton provides the shape and the barrier layer provides the barrier that prevents flow between the left atrium and the LAA. In use, the balloon from the balloon implant system, when inflated, opens the endoskeleton from a collapsed state to an implant or open state. When fully implanted, the balloon is then deflated. The deflated balloon and associated catheters are removed from the atrium.

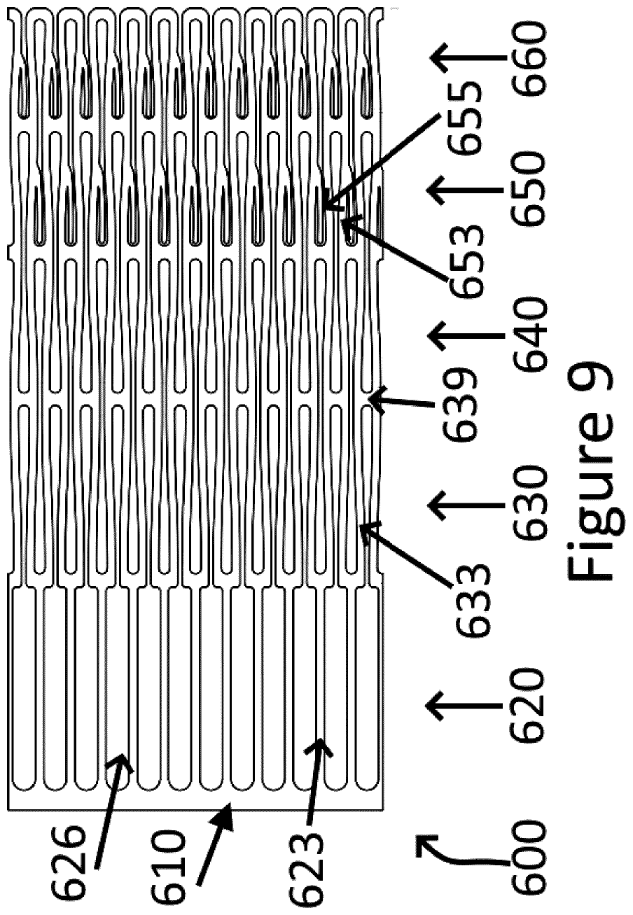

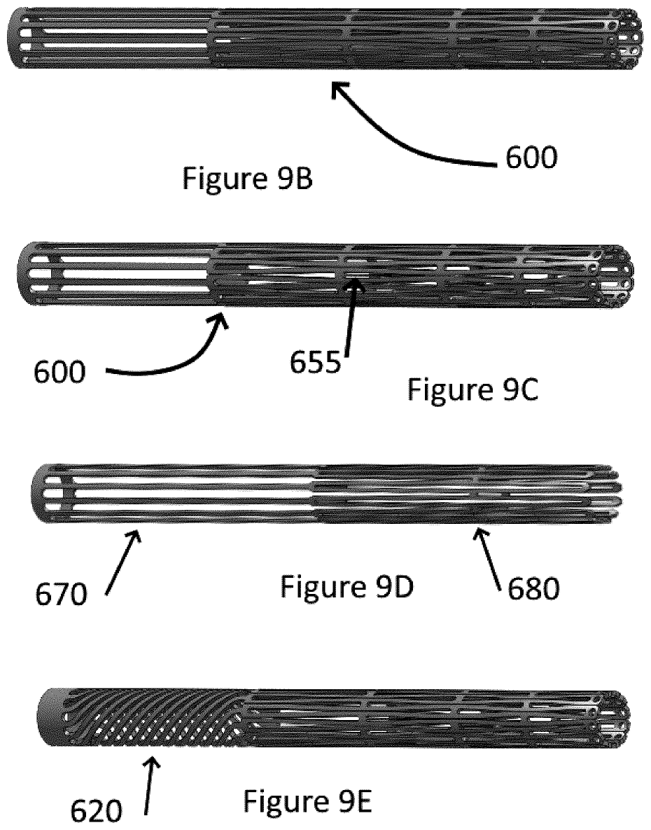

Construction of the implant system begins with the endoskeleton. As shown in FIG. 9, the endoskeleton can be constructed by laser cutting a suitable material into a suitable pattern. While the pattern, e.g., the pattern shown in FIG. 9, may be cut into a flat material, in a preferred embodiment a hypotube 600 is first provided. A portion of the 2D pattern shown in FIG. 9 is laser cut into the hypotube. The hypotube is rotated in 1/12 sections, cut, and rotated again until the entire pattern is cut.

While other materials are contemplated, stainless steel, titanium, and tungsten are preferred materials. The material used must have an appropriate combination of elastic modulus, elongation, and hoop strength. The elastic modulus must be low enough that the compliant balloon can provide the stress necessary to push the endoskeleton past its elastic deformation stage and into plastic deformation by crossing its elastic limit or yield strength. The patterned hypotube 600 must also have enough stretchability it does not break, and a high enough hoop strength to hold its position in the LAA.

In an exemplary embodiment, the balloon is constructed of silicone, polyurethane, or nylon with a Shore A hardness of 70-90. In this case the balloon may provide a pressure of 2 atmospheres. In another embodiment the balloon can provide pressures between 1-4 atmospheres.

Thus, the elastic modulus of the material must range between 50-700 GPa. Preferably the elastic modulus of the hypotube material ranges between 100-450 GPa. Suitable materials would include stainless steel, with a elastic modulus between 100-310 GPa. One type of suitable stainless steel would include the 300 series, with a modulus between 175-210 GPa. Another suitable material would be titanium with an elastic modulus around 113 GPa. However, titanium is not as stretchable prior to breaking as stainless steel. Tungsten with an elastic modulus around 400 GPa is also suitable.

The yield strength for the material should range from 75 MPa to 1000 MPa, preferably between 100 MPa and 800 MPa. 300 series stainless steel, for example, has a yield strength of 215 MPa, Titanium a yield strength of 140 MPa, and Tungsten a yield strength of 750 MPa.

As described below, the hypotube is sufficiently thin enough and flexible enough to be deformed outward by inflation of a compliant balloon. The materials above, under stress, will undergo plastic deformation and remain in an open configuration even upon withdrawal of the balloon.

As shown in FIG. 9, endoskeleton 600 can comprise different sections. Proximal ring or band 610 provides a first, largely non expandable portion at the proximal end. Upon expansion of endoskeleton 600, the ring 600 will form the middle or center of the implant. Second portion 620 comprises narrow bands 623, with large open areas 626. The number of bands 623 may vary depending on the size needed, and can range from 4-20, e.g., 8-16, or 12 as shown. By providing a second portion 620 with very little material present, this section will be very flexible and will expand very easily.

Third portion 630 provides tines 633 extending in the axial direction. As shown in FIG. 9A, a first tine 633′ and a second tine 633″ are joined at their proximal side by a connection 636. However, at their distal end, they are not joined. Rather, second tine 633″ and third tine 633′″ are joined by a connection 639. Thus, the serpentine pattern of the second portion 620 allows for relatively easy expansion, but remains strong, and creates an X pattern upon opening.

In the embodiment shown in FIG. 9, fourth portion 640 is constructed similarly to third portion 630. Embodiments with neither 630 or 640; only 1 such portion (e.g., 630, but no 640) or many such portions-three or more are contemplated. For example, section 620 may be omitted and a third portion 640′ may be added.

Fifth portion 650 provides a denser section. In particular, tines 653 are shorter than tines 643, and as such resist opening more. Fifth portion 650 further includes barbs or hooks 655. Upon opening, barbs 655 will extend away from the endoskeleton for piercing the LAA. Sixth section 660 is constructed similarly to fifth section 650, and as above the number of such sections may be varied. FIG. 9B shows the endoskeleton 600 in 3D form.

Other construction styles are contemplated. For example, FIG. 9C shows a similar endoskeleton style, but with barbs 655 that are connected to the endoskeleton 600 on their proximal side and free on their distal side, rather than connected to the endoskeleton 600 on their distal proximal side and free on their proximal side as in FIG. 9A. FIG. 9D shows an endoskeleton with two sections, a first longer section 670, and a second, distal, section 680. The increased length of the cutouts in the first section will allow the endoskeleton in 9D to open more easily. FIG. 9E shows an endoskeleton with a spiral cutout pattern in first section 620.

If the hypotube is constructed of a single material, such as stainless steel 304 or another 300 series stainless steel, the nature of the endoskeleton's expansion is controlled by the material removed. In those sections where more material is removed, the expansion will be easier. Likewise, longer struts with fewer connections will allow for more expansion. Thus, the shape of the endoskeleton as expanded will be controlled by the pattern cut in the hypotube. The pattern shown in FIG. 9 will allow for easy expansion in the proximal portions, but also allows for the endoskeleton to assume an irregular form and fit the LAA anatomy. Because the balloon is compliant, at its expansion it would readily assume whatever shape the LAA portion it reside in takes. The pattern of FIG. 9 likewise allows the endoskeleton to take irregular shapes, unlike the prior art which assumes a preset shape upon release from the catheter.

In the prior art the implant is typically formed of a shape memory wire, and typically assumes a circular open shape upon release. That is, it is the implant is not forced open and into the LAA by a balloon, as in the present case, but rather is been heat set to a particular open shape, and once it is unconstrained it assumes that shape. That shape is typically circular, and these circular shapes do not account for the actual geometry of the LAA. The present invention, by using a compliant balloon to open a semi compliant endoskeleton allows the implant to assume a form that completely occludes the LAA ostium, whether that form be circular, oval, or otherwise irregular.

In another embodiment, the hypotube may be constructed of multiple materials. Thus, the proximal end may be constructed of a lower modulus stainless steel, and the distal end may be constructed of a higher modulus titanium (or higher modulus stainless steel). In this fashion the open geometry may also be controlled.

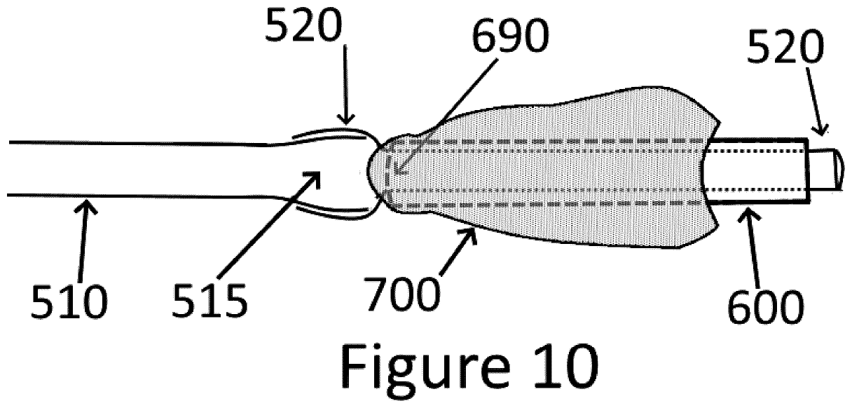

Once the endoskeleton has been laser cut, the barrier layer must be attached. As shown in FIG. 10, a small pore compliant barrier layer 700 is attached to endoskeleton 600. In FIG. 10 the barrier layer 700 is attached outside of endoskeleton 600, but it may be inside as well, depending on application.

In a preferred embodiment the barrier layer 700 is constructed of a knit fabric with very small pore sizes. The pore cross section dimension is preferably in the range of 0.00003 sq.in. to 0.00008 sq.in., e.g., 0.000041 sq.in. The material is PET (Polyethylene terephthalate). These dimensions will allow some blood flow between the LAA and the LA, but will stop emboli.

The fabric is compliant in both the x and y directions, allowing it to take on the irregular shapes of the LAA. The fabric may be bunched around the endoskeleton, allowing it to unfold as the endoskeleton is opened, or it may simply be stretchable. The fabric may be attached to the endoskeleton 600 by any means, such as by sewing it in place.

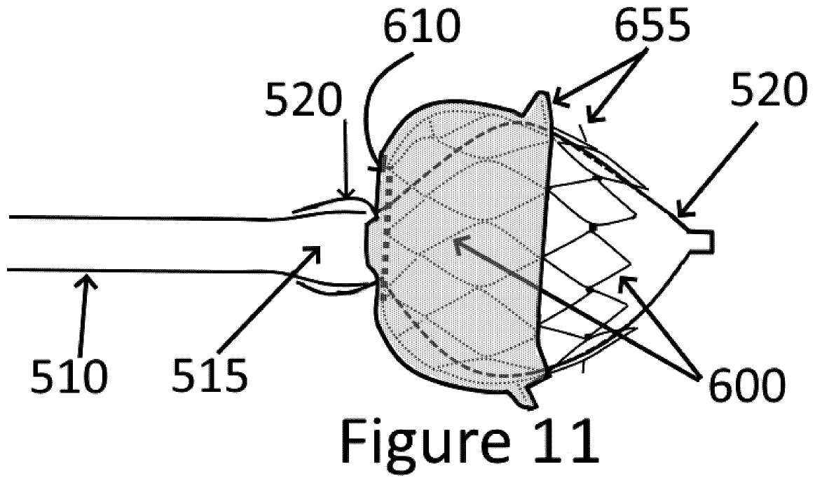

Once manufactured, the implant system is slid over the balloon system from the distal end. In the embodiment shown in 8A, the outer catheter 510 has a larger diameter at its distal portion 515 than the portion of the balloon 520 next to it. In other embodiments the balloon will have a larger diameter. In either case, the diameter of the outer catheter 510 or an accompanying sheath is chosen such that it is sufficiently larger than the lumen 690 of the endoskeleton. As such, the endoskeleton can be slid over the balloon 520, but will stop when it reaches the outer catheter 510. The balloon 520 has a diameter that is close to that of the endoskeleton's lumen, and as such friction will hold the endoskeleton 600 in place. The endoskeleton's lumen can pass over the middle of balloon 520, but not readily, and as such is held against the distal end 515 of catheter 510, as shown in FIGS. 10-11.

While a friction fit is described above, other types of securement methods are possible. The lumen of the endoskeleton 600 may be larger enough to pass over a first portion of the catheter, but small enough it is held in place there. The catheter's distal end may also have an interference feature that holds the endoskeleton's lumen in place. While it is not as advantageous for the procedure as a friction fit, the implant system may include a threaded fitting, and be screwed in place. However, a threaded fitting can be a disadvantage as it leaves open a metal object in the heart, and can be a location for emboli formation, as well as the last portion of the surface to endothelialize. A detent may also be used to hold it in place.

The implant system and the balloon implant system, once assembled, are maneuvered through the sheath and over the guidewire to the LAA while in a collapsed or deflated status, as shown in FIG. 10. Various means can be used to determine when the device is in position. Radiopaque markers and fluoroscopy can be used to determine location. If the radiopaque markers are on the balloon, for example, or on the endoskeleton or barrier, they may be used to determine location.

Once in place balloon 520 is inflated, as shown in FIG. 11. Inflation of the balloon forces the endoskeleton 600 and the barrier layer 700 outward. Ideally, the balloon 520 is inflated according to the amount of inflation found by the sizing balloon, earlier. Barbs 655 pierce the LAA tissue, holding the implant in place. Imaging technologies such as transesophageal echocardiogram (TEE) and/or Intracardiac echocardiography (ICE) can be used to determine if the implant is fully occluding the LAA ostium. Additional fluid volume is added to the sizing balloon until the TEE or ICE imaging confirms full closure of the LAA ostium.

The Endoskeleton 600's segment length and geometry are designed to expand when exposed to stresses provided by inflating the balloon. As the stress is applied to the structure, it causes the material to undergo strain. Stainless steel is a material that can undergo plastic deformation. Plastic deformation is the permanent distortion that occurs when a material is subjected to tensile, compressive, bending, or torsion stresses that exceed its yield strength and cause it to elongate, compress, buckle, bend, or twist. In plastic deformation permanent changes occur with in the material itself.

This plastic deformation is the mechanism that enables the endoskeleton to remain in the expanded geometry even once the stress (inflated balloon) is removed. The segment 620 nearest the proximal end of the endoskeleton requires the least amount of stress to expand and each adjacent segment (630, 640 moving to 650, 660) moving toward the distal end of the endoskeleton requires an increasing amount of stress to expand. This variation in the stress required to expand and the geometry of the segments has been designed such that the maximum diameter of the deployed device (the maximum diameter is the high end of the size range) is at roughly 60% of the device's overall length (Portions 620, 630, 640). The distal most 40% of the endoskeleton segments (portions 650, 660) won't stretch as much thus keeping them directionally pointed toward the axis of the endoskeleton once deployed thus preventing unintended perforations of the LAA.

The mechanical features (barbs) located in the portions 650, 660 that are angled outward such that they grab a hold of the LAA tissue and provide an anchor for the deployed device are located very near the maximum diameter of the deployed device.

The anatomy of the LAA can be very irregular. That is, the opening is not formed in a perfect circle. The prior art devices typically use a nitinol structure that is heat set to a specific “round” geometry. When deployed, the nitinol structure can only take the round shape that was defined by the heat setting step. When a prior art device is placed in the LAA, and released, it will only pop open to a predetermined shape, and it will seek to return to that shape if unconstrained. If the ostium of the LAA is not round, which almost is always the case, there is a chance that there will be some portion of the ostium that is not closed off.

The present invention solves this issue. First, by using a compliant balloon material such as polyurethane, the balloon is soft enough to expand and deform to the shape it is being expanded into, specifically the opening of the LAA.

The endoskeleton is designed to be semi-compliant. The endoskeleton is compliant enough to deform to the same geometry as the balloon during the balloon inflation, but it also has sufficient hoop strength to maintain its shape once it is expanded. The compliancy of the structure allows it to more readily take an irregular shape than if it was heat set to a predefined geometry. The maintaining of the shape along with the mechanical holding elements (barbs) keep the implanted endoskeleton inside the ostium of the Left Atrial Appendage (LAA). Additionally, the metal endoskeleton structure is partially covered with a fabric that is compliant in both the X and Y axis, which also means it is compliant on the bias of these 2 axes.

Likewise, barrier layer 700 is constructed of a knit fabric with very small pore sizes. The fabric is compliant in both the x and y directions, allowing it to take on the irregular shapes of the LAA.

One closure has been confirmed, and ideally a successful implant is confirmed, the balloon is deflated. In a preferred embodiment it is deflated to a vacuum, to make removal from the endoskeleton lumen easier. In another embodiment the delivery sheath is advanced to the proximal end of the endoskeleton implant and held in place. While holding the sheath against the implant, the implant balloon is pulled proximally out of the endoskeleton.

Once the implant balloon is deflated, the catheter 510 may be removed. At this point, as the endoskeleton has been expanded and has deformed to match the LAA anatomy, and as the barbs are in place in the LAA tissue, the implant is held in place sufficiently strongly to overcome the friction hold. As a result, ring 610 slides over balloon 540 and catheter 510, and is disengaged from the remainder of the device, remaining in place in the LAA.

Preferably, the lumen of the endoskeleton includes a valve, such as a silicone valve (not shown) that will close as the catheter and balloon are removed from the lumen.

Once the implant system is disengaged, the delivery system and balloon implant system are removed. The presence of the small pore compliant fabric on the surface of the metal endoskeleton prevents emboli from moving from the Left Atrium (LA) into the LAA, and vice versa. During the first 30+ days after implant the body will create an endothelium across the surface of the fabric permanently closing off the ostium to the LAA.

1. An implantable device adapted for left atrial appendage occlusion, comprising:
a plurality of elongate members each having a distal end, wherein the distal ends are not directly coupled to anything, and optionally wherein at least one of the distal ends is atraumatic.

2. The implantable device of claim 1, wherein each of the plurality of elongate members is not directly coupled to anything, and does not have any branching members, along at least 50% of its length, measured from the distal ends of the elongate members.

3. The device of claim 1, wherein the plurality of elongate members have proximal ends that are coupled, either directly or indirectly, to a proximal end of at least one of the other proximal ends.

4. The device of claim 1, wherein the plurality of elongate members taper generally in the distal direction, and optionally which together define a general pear shape.

5. The device of claim 1, wherein the plurality of elongate members each have proximal regions that are coupled to a LAA barrier.

6. The device of claim 1, wherein the plurality of elongate members each have proximal regions that extend distally when in a non-expanded configuration, and radially inward after the implantable device is expanded.

7. The device of claim 1, wherein the plurality of elongate members each have proximal regions that are adapted to deform from a first delivery configuration to a second barrier configuration.

8. The device of claim 1, wherein the plurality of elongate members do not form a stent.

9. The device of claim 1, wherein the plurality of elongate members are not formed by laser cutting a tubular member.

10. A system for positioning a balloon expandable LAA occlusion device, comprising:
an elongate shaft carrying an expansion balloon, the elongate shaft having an inflation lumen therein in fluid communication with an interior of the balloon;
an implantable LAA occlusion device adapted to be fit over at least a portion of the balloon in a non-expanded configuration, the implantable device including a plurality of balloon expandable elongate members, the elongate members having open ends and proximal ends attached to a barrier.

11. The system of claim 10, wherein the proximal ends assume a flatter configuration that forms a proximal region of the implant when the device is expanded.

12. A method of occluding a left atrial appendage (“LAA), comprising:
delivering a system for deploying a left atrial appendage occlusion device to the left atrial appendage, the system comprising:
a delivery catheter, the delivery catheter comprising:
a balloon attached to a distal end of the delivery catheter; the balloon having a preformed shape when inflated; wherein the balloon is constructed of a compliant material;
a left atrial appendage occlusion device comprising:
an endoskeleton, the endoskeleton constructed of a flexible material and comprising barbs;
a barrier layer;
inflating the balloon from a deflated form to the preformed shape;
expanding the endoskeleton with the inflated balloon;
plastically deforming the endoskeleton from a first, compressed form to a second, expanded form;
deflating the balloon;
removing the balloon and the delivery catheter;
wherein the endoskeleton is configured to remain in the second, expanded form.

13. The method of claim 12, wherein the endoskeleton further comprises barbs, and further comprising the step of pushing the barbs into the left atrial appendage.

14. The method of claim 12, wherein the endoskeleton is comprised of a material with an elastic modulus between 100-310 GPA.

15. The method of claim 14, wherein the hypotube has a pattern cut into it, the pattern configured to allow the hypotube to expand outward under pressure from the balloon.

16. The method of claim 12, further comprising the step of injecting contrast media to identify the presence of blood flow between the left atrium and the left atrial appendage.

17. The method of claim 12, further comprising the steps of inserting a sizing balloon into the left atrial appendage;
inflating the sizing balloon with a known amount of inflation media;
deflating the sizing balloon;
removing the sizing balloon;
using the known amount of inflation media to calculate the size of the left atrial appendage.
